(19) United States
(12) Patent Application Publication
(10) Pub. No.: US 2020/0281820 A1
(43) Pub. Date: Sep. 10, 2020

(54) MEDICAL PROBE INSERTION SYSTEM

(71) Applicant: HAEMOBAND SURGICAL LIMITED, Belfast (GB)

(72) Inventor: Essam GHAREEB, Enniskillen (GB)

(21) Appl. No.: 16/756,461

(22) PCT Filed: Oct. 15, 2018
(86) PCT No.: PCT/EP2018/078038

§ 371 Date: Apr. 15, 2020

(30) Foreign Application Priority Data
Oct. 16, 2017 (GB) 1716962.4

(51) Int. Cl. A61J 15/00

(52) U.S. Cl. CPC A61J 15/0088; A61J 15/0003; A61B 5/065; A61M 2205/582; A61M 2205/584

(57) ABSTRACT

A probe insertion system comprising an elongate flexible probe such as a nasogastric tube. The probe has at least one bend sensor for detecting bends. The probe comprises a probe body having at least two longitudinally spaced, visually distinct body segments. The system further comprises means for rendering to a user an indication of a detected bend.

### FIELD OF THE INVENTION

This invention relates to the insertion of medical probes particularly, but not exclusively, nasogastric tubes.

### BACKGROUND TO THE INVENTION

Nasogastric (NG) tube insertion is one of the most practiced procedures in health care. For many years NG tubes have been inserted through a patient's nose into the stomach, for different indications. For example, in emergency situations NG tubes are used to evacuate the stomach in cases of intestinal obstruction, persistent vomiting, gastric dilatation or torsion, gastric outlet obstruction, or upper GIT bleeding, and in medical conditions as cerebrovascular accidents, or during loss of consciousness due to prevent aspiration of gastric contents into the lungs. NG tubes are also used as feeding tubes.

A problem with introducing an NG tube into a patient's body is the significant risk that it may go into the trachea, bronchi and lungs or curl up in the oesophagus. In such instances, giving feeds or irrigating the tubes may result in fluid entering the lungs, or may affect aspiration, which can be fatal. It is therefore essential to confirm the correct location of the tube in the stomach before use.

Many methods have been proposed for confirming the location of NG tubes, including using litmus paper to check the acidity of the sucked gastric contents, injecting air into the tube and listening to bubbling sounds over the stomach, or taking an X-ray image of the abdomen. X-ray imaging has proved to the most reliable method, as it detects an X-ray marker in the tube. However, X-ray images can be difficult to interpret due to the faint lines and the presence of many other shadows made by other leads and tubes used on the patient. Also, it is recommended that prior to every irrigation or introduction of any feeds to the patient that an X-ray is obtained to confirm the position of the tube in the stomach. This incurs significant costs in terms of equipment, time and personnel, as well as exposing patients and staff to X-rays frequently.

It would be desirable to provide an improved system and method of confirming correct location of an NG tube.

### SUMMARY OF THE INVENTION

A first aspect of the invention provides a probe insertion system comprising an elongate flexible probe having at least one bend sensor for detecting bends in at least one portion of the probe, the probe comprising a probe body having at least two longitudinally spaced, visually distinct body segments, the system further comprising means for rendering to a user an output indicative of at least one detected bend. Preferably, said at least one portion comprises a leading end portion.

At least two of, or all of, said body segments may be arranged contiguously along the probe.

Alternatively, at least two of, or all of, said body segments are longitudinally separated.

Preferably, each body segment is visually distinguishable from at least the, or each, nearest other body segment. Each body segment may be visually distinguishable from each other segment.

Typically, at least one of said body segments is visually distinguishable by at least one visual indicator. At least one of, and optionally all of, said body segments may be visually distinguishable by its colouring. At least one of, and optionally all or, said body segments may be visually distinguishable by its patterning. At least one of, and optionally all of, said body segments may be visually distinguishable by its texturing.

In preferred embodiments, at least one of, and optionally all of, said body segments is configured, by its position on the body and/or its length, for exposure at a patient ingress point in a designated insertion circumstance during use.

Said rendering means may be configured to render to the user an output indicative of any one or more of: a detected presence of a bend; detection that a bend exceeds a threshold level; and/or detection of a loop in said body.

Said rendering means may be configured to render to the user an output indicative of at least one bend detected in a leading end portion of the body.

Said rendering means may comprise at least one output device coupled to said at least one bend sensor. For example said at least one output device may comprise an audio, visual and/or haptic output device.

Said at least one output device may comprise a computing device. Said computing device may be configured to render an image of the probe including any detected bends.

In preferred embodiments, each of said body segments is at least 2 cm long, preferably between 2 cm and 40 cm long, and most preferably between 5 cm and 30 cm long.

In some embodiments, said at least two body segments comprise first, second and third body segments, said first segment being closest to a leading end of the probe, the third body segment being farthest from the leading end and the second body segment being between said first and third body segments.

Said first body segment may be between 10 cm-20 cm in length, preferably approximately 15 cm in length. Said second body segment may be between 15 cm-25 cm in length, preferably approximately 20 cm in length. Said third body segment may be between 20 cm-30 cm in length, preferably approximately 25 cm in length. Said first, second and third body segments may be contiguously arranged on said probe.

At least part of said first body segment may be located approximately 10 cm-20 cm from the leading end of the probe. At least part of said second body segment may be located approximately 30 cm-40 cm from the leading end of the probe. At least part of said third body segment may be located approximately 55 cm-65 cm from the leading end of the probe.

In preferred embodiments said probe is a nasogastric tube. The preferred system is a nasogastric tube insertion system. From a second aspect the invention provides an elongate flexible probe having at least one bend sensor for detecting bends in at least one portion of the probe, the probe comprising a probe body having at least two longitudinally spaced, visually distinct body segments.

In preferred embodiments, the NG tube comprises at least one bend sensor (also known as flexion sensors), incorporated into the tube structure by any suitable means, and capable of detecting bends in a respective portion of the tube. Signals generated by the, or each, sensor may be used to inform an operator of bends in the respective portion(s) of the tube, especially at a forward portion of the tube. Any suitable type of sensor may be used, for example a conductive ink-based bend sensor or a fibre optic sensor or a conductive fabric-based, conductive thread-based or conductive polymer-based sensor.

In addition to the bend sensor(s), the preferred tube is visually segmented along its length, i.e. comprises at least two, and optionally three or more, lengthwise, or longitudinally spaced-apart, segments that are visually distinguishable from one another when viewed by the operator. The segments are typically contiguous with one another along the length of the tube, although may alternatively be separated from one another along the length of the tube. In this context, the word “longitudinally” means along the length, or lengthwise, of the tube, i.e. the end-to-end direction.

The length of each segment may be the same or different. Advantageously, the length and/or longitudinal location of each segment is determined by the anatomy of the target patient, e.g. a human adult or a human child. In particular, the segments may be arranged to correspond to respective aspects of the patient's anatomy between the nostril and the stomach, i.e. the intended ingress location and end location of the tube during use. Advantageously the length and/or longitudinal location of each segment is determined by a respective location of the patient's anatomy where the tube might bend.

In preferred embodiments a first segment is provided at, or closest to, the leading end (or tip) of the tube and its longitudinal position on the tube with respect to the leading end is determined by the distance between the nostril and the oral pharynx. A second segment may be provided, further from the leading end than the first segment, its longitudinal position on the tube with respect to the leading end being determined by the distance between the nostril and the lower end (carina) of the trachea. A third segment may be provided, further from the leading end than the first segment and the second segment, its longitudinal position on the tube with respect to the leading end being determined by the distance between the nostril and the stomach. The arrangement is such that, when the leading end of the tube is in the vicinity of the oral pharynx, at least part of the first segment of the tube is visible at the patient's nostril (i.e. it is the first segment of the tube that is protruding out from the nostril). However, should the leading end of the tube reach the lower end of the trachea, then at least part of the second segment of the tube is visible at the patient's nostril (i.e. it is the second segment of the tube that is protruding out from the nostril). When the leading end of the tube reaches the stomach, then at least part of the second segment of the tube is visible at the patient's nostril (i.e. it is the third segment of the tube that is protruding out from the nostril).

For example, in a typical embodiment, the tube may have a first segment of approximately 15 cm in length located at its leading end. A second segment may be provided, further from the leading end than the first segment, of approximately 20 cm in length. A third segment may be provided, further from the leading end than the first segment and the second segment, of approximately 25 cm in length. In this example the segments may be contiguous such that the first 15 cm of the tube (measured from its leading end) provides one segment, the next 20 cm of the tube provides the second segment and the following 25 cm provides the third segment.

By correlating detected bends with whichever segment of the tube is protruding from the nostril (or other point of tube ingress), the user can determine if the tube has been correctly or incorrectly inserted.

Preferred embodiments of the invention provide reliable means for confidently informing an operator of the position of the NG tube without requiring complicated equipment, personnel or procedures, as well as being cost effective. Preferred embodiments are simple to use and are therefore suitable for use by junior doctors and nurses, even outside of a hospital environment. Advantageously, complicated electronics for measuring the inserted part of the tube are not required, which facilitates ease of use and low costs, and reduces the risk of spreading infections.

Further advantageous aspects of the invention will be apparent to those ordinarily skilled in the art upon review of the following description of a specific embodiment and with reference to the accompanying drawings.

### BRIEF DESCRIPTION OF THE DRAWINGS

Embodiments of the invention is now described by way of example and with reference to the accompanying drawings in which like numerals are used to denote like parts and in which:

FIG. 1 is a schematic diagram of a probe insertion system embodying one aspect of the invention;

FIG. 1A is a longitudinal cross-section view of a probe, the probe being part of the system of FIG. 1;

FIG. 2 shows a probe, in the form of a nasogastric tube, embodying another aspect of the invention and suitable for use in the system of FIG. 1; and

FIG. 3 shows an alternative probe, also in the form of a nasogastric tube, embodying the invention.

### DETAILED DESCRIPTION OF THE DRAWINGS

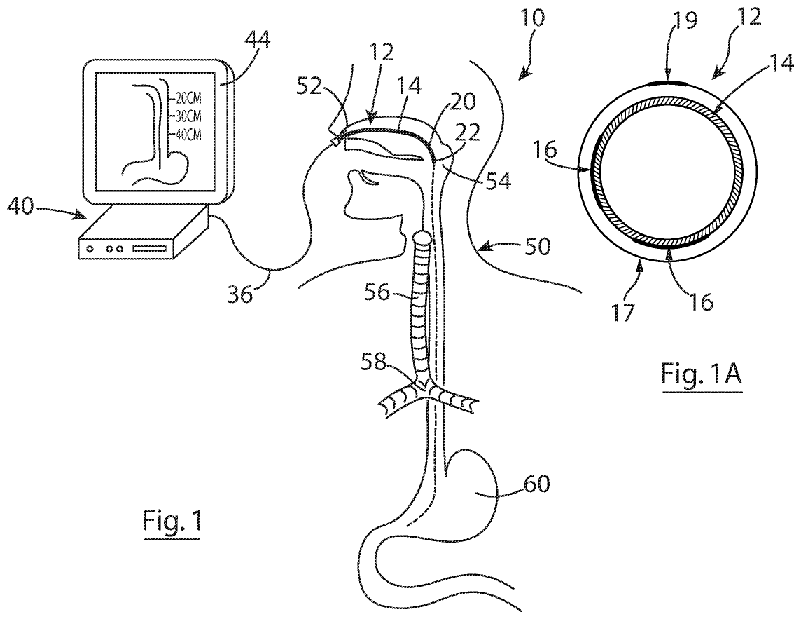

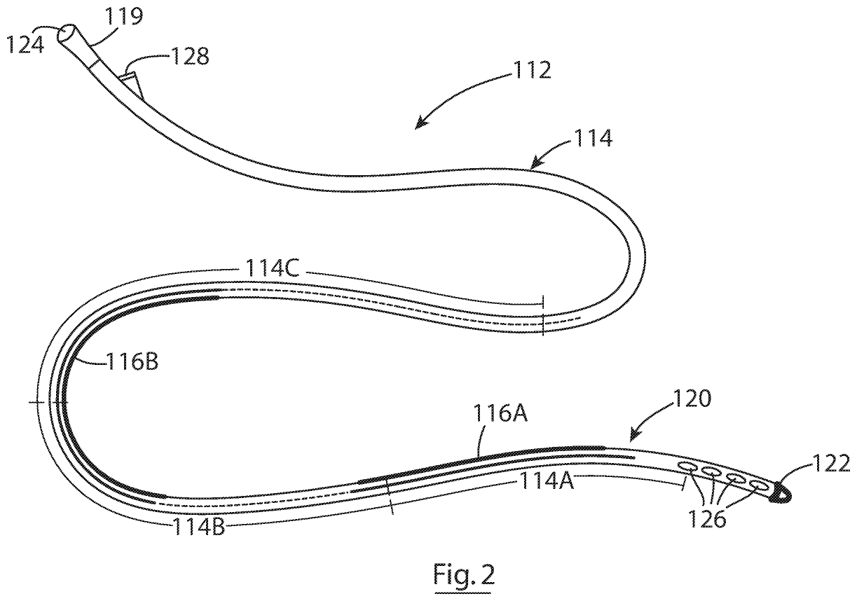

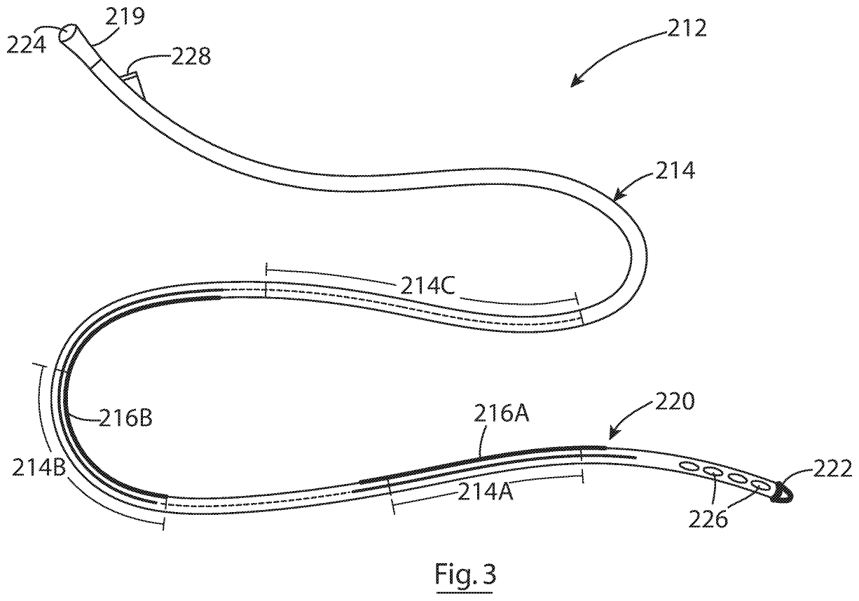

Referring now to the drawings, there is shown generally indicated as 10 a probe insertion system embodying one aspect of the invention. The system 10 includes a probe 12 embodying another aspect of the invention. Embodiments of the probe are shown in more detail in FIGS. 2 and 3. The probe 12 comprises an elongate flexible body 14, which may be formed from any suitable material typically plastics or a dielectric material. The body 14 may have any cross-sectional shape although it is typically substantially circular in cross-section, as for example is shown in FIG. 1A. Alternatively, the body 14 may comprise a substrate having a substantially rectangular, or otherwise polygonal, cross-section defining substantially flat surfaces on each face of the body 14. The body 14 is flexible (and preferably non-resilient) advantageously to the extent that it can bend freely during use to conform to the shape of any passage into which it is introduced. For example, the body 14 may be made of a material that allows it to bend in two mutually perpendicular planes, or to bend in a first plane and to be rotatable (or twistable) about an axis in that plane to allow it to bend in a plane that is perpendicular to the first plane.

In preferred embodiments, the probe 12 comprises a tube, especially a nasogastric tube. In alternative embodiments, the probe 12 may be intended for use as a catheter, in which case the body 14 may be hollow, having one or more lumen 13 to allow fluids to pass through it or to allow another probe (not shown), for example an endoscope or other instrument, to be fed through it. In other embodiments, the probe 12 may itself be intended for use as an endoscope or other instrument, in which case the body 14 may be solid or hollow as required by the application and may incorporate other devices, e.g. a camera and/or a lamp (not shown) as required. Alternatively still, the probe 12 may be located, in use, in a channel or passage formed in another probe, e.g. an endoscope or other instrument. In preferred embodiments, therefore, the system is a medical probe insertion system and the probe is a medical probe. However, it will be understood that embodiments of the invention may have non-medical applications.

At least one bend sensor 16 is provided on the body 14 in order to detect bends in the body 14. In the embodiment of FIG. 1, a plurality of bend sensors 16 are provided on the body 14. Each bend sensor 16 may provided on an outer surface of the body 14, preferably covered by one or more sheath or cover 17 which is fitted over the body 14 and may extend wholly or partly along the length of the body 14, preferably at least being long enough to cover the sensors 16. The sheath may be formed from any suitable material, e.g. rubber or plastics. The sensors 16 may be fixed in position by any suitable means, e.g. adhesive. Alternatively, the sensors 16 may be embedded in the body 14, or provided on an internal surface of the body 14. One or more X-ray markers 19 may be provided on the body 14, for example on the cover 17.

The probe 12 includes means for producing an output signal that is indicative of at least one characteristic of a detected bend the body. Typically this is provided by an electrical, optical or electro-optical circuit (not shown) forming part of the bend sensor and/or to which the bend sensor 16 is connected.

Each bend sensor 16, when incorporated into a suitable circuit, causes the circuit to generate an electrical, optical or electro-optical output signal, as applicable, that is indicative of one or more characteristics of a bend in the portion of the body 14 at which the sensor 16 is located. For example, the output signal may be indicative of the extent of the bend (i.e. the relative sharpness of the bend) and/or the direction of the bend (i.e. whether it is convex or converse with respect to a notional reference point). One or more thresholds may be defined for determining when a bend is part of a loop. For example, the extent of a bend (i.e. the relative sharpness of the bend) as determined from the output signal may be compared against one or more threshold value, and depending on the result, e.g. if a relevant threshold is exceeded, then the presence of a loop is determined.

In preferred embodiments, each bend sensor 16 comprises a sensing component having an electrical or optical property, conveniently electrical resistance, that changes depending on how the sensing component is bent. The sensing component is incorporated into a circuit that produces an output signal that is dependent on the value of the resistance, or other relevant electrical or optical property, of the sensing component. Advantageously, the circuit is configured such that the output signal continuously indicates the extent to which the component is bent, i.e. is continuously indicative of the relevant electrical property, typically resistance, of the component.

The sensing component typically comprises an elongate strip and may comprise any suitable bend-sensitive material, for example a piezoresistive material (typically semiconductor materials such as germanium, polycrystalline silicon, amorphous silicon, silicon carbide, or single crystal silicon), carbon or a conductive fabric or a conductive or resistive ink (any of which may provided on or in a substrate if necessary, e.g. on a surface, especially a flat surface, of the body 14). Optionally, each sensing component may comprise two or more sensing components, of any suitable type including those described above, electrically or optically connected in series. Optionally, where the sensing component comprises a strip of bend-sensitive material, one or more electrically conductive pads may be provided along its length (spaced-apart where there is more than one) in order to control, and typically reduce, the electrical resistance of the strip. Alternatively, the, or each, bend sensor may be a fibre optic bend sensor.

Typically, each bend sensor 16 has an axis along which it is sensitive to being bent (i.e. to produce the change in electrical or optical property in this example). Where the sensing component is elongate, the bend-sensitive axis is typically the longitudinal axis. In use, the bend sensor 16 is therefore capable of detecting bending of the object on which it is located in a plane in which the bend-sensitive axis lies.

Conveniently, each bend sensor 16 is located on the body 14 (e.g. mounted on its surface or embedded therein), and connected to the respective circuit by conductive wire(s), which may take the form of conductive tracks provided on a substrate (e.g. the body 14), or light guide(s) as applicable, either of which may be provided on or embedded in the body 14 as is convenient. In some embodiments, the respective circuits are located remotely from the bend sensor 16, for example at the proximal end of the probe 12 or separately from the probe 12. Alternatively, the respective circuit may be co-located with the bend sensor 16 on the body 14. In FIG. 1, the sensors 16, and any associated circuitry as applicable, are connected to other components of the system 10 (by cable 36 in this example), as described in more detail hereinafter. In alternative embodiments, any suitable connector (not shown) may be provided for allowing connection to other components of the system 10. In alternative embodiments the bend sensors 16 on the body 14 may be equipped to communicate wirelessly with the insertion system 10.

The system 10 of FIG. 1 comprises a monitoring device 40 that includes means for processing the signals received from the sensors 16, preferably in order to create a data model representing the shape of the probe 12 in three dimensions. This may be achieved combining the respective output signals from the sensors 16 of each set with an indication of each set's longitudinal location on the body 14. The monitoring device 40 preferably also includes means for rendering the data model as an image on a VDU 44, which may be part of or otherwise connected to the monitoring device 40. The monitoring device 40 conveniently comprises a computer running suitable computer software for creating the data model and preferably also rendering the image. The data model may be created using any convenient conventional techniques and may be configured to use the bend information determined from the output signals generated by the sensors 16. Loops in the probe 12 may be identified by comparing the relative sharpness of one or more detected bends against one or more threshold values. The respective outputs from more than one sensor 16 may be analysed, e.g. aggregated or otherwise collectively assessed, to determine on which side the probe 12 crosses itself in a detected loop.

The cable 36 may be configured to deliver electrical power to the sensors 16, if required, the power being supplied by the monitoring device 40 for example. Alternatively, a separate electrical power source (not shown) may be provided for the probe 12, typically being connectable to the cable 36 or otherwise to the wires.

In the embodiment of FIG. 1, the monitoring device 40 serves as an output device providing means for rendering to the user (preferably in the form an image of the probe 12) an indication of detected bends. More generally, the rendering means comprises at least one output device coupled to the, or each, bend sensor 14 and being capable of rendering bend information to the user, for example as audio, visual and/or haptic output. For example, in simple embodiments it is not essential for the user to see an image of the probe, but only to know when a bend has occurred. As such, the output device may comprise any conventional audio, visual and/or haptic output device, such as a lamp, a buzzer and/or a vibrator, or any output device capable of rendering a suitable output signal to the user.

The probe 12 may have multiple bend sensors 16, or multiple sets of bend sensors, spaced apart along its length. This facilitates creating an image of the probe 12 as described above. In alternative embodiments, it is only necessary to detect when a bend has formed in one portion of the body 14, preferably an end portion 20. The end portion 20 is the portion of the body 14 that is adjacent the tip 22 of the probe 12 that is first inserted into a patient 50 during use, i.e. the leading end of the body 14. In alternative embodiments therefore only one bend sensor 16, or one set of bend sensors 16, may be provided, located at the leading end portion 20.

FIGS. 2 and 3 show respective embodiments of the probe 112, 212 in the form of a nasogastric tube. The same or similar description applies in relation to the nasogastric tubes 112, 212 of FIGS. 2 and 3 as is provided in relation to the probe 12, and vice versa, like numerals being used to denote like parts.

The probe 112, 212 comprises an elongate flexible body 114, 214 having a first port 124, 224 at one end 119, 219, and a second port 126, 226 at the other, leading, end 120, 220, typically adjacent the tip 122, 222. In the illustrated examples, the second port 126, 226 comprises multiple apertures. The first and second ports 124, 224, 126, 226 may serve as fluid inlets or fluid outlets depending on the application of the tube 112, 212. A third port 128, 228 may be provided, for example for the introduction of a syringe. The ports are in fluid communication with each other via one or more lumen.

The probe body 114, 214 has at least two longitudinally spaced, visually distinct body segments, denoted in FIGS. 2 and 3 as 114A, 1148, 114C and 214A, 214B, 214C respectively. In this context longitudinally means along the length, or lengthwise, of the body 114, 214, i.e. its end-to-end direction.

All of the body segments 114A, 1148, 114C may be arranged contiguously along the body 114, i.e. are longitudinally spaced but arranged end-to-end (as for example is shown in FIG. 2). In alternative embodiments the visually distinct segments 214A, 214B, 214C may be longitudinally separated from each other (as for example is illustrated in FIG. 3), or some segments may be contiguous while others are longitudinally separated.

Each body segment 114A, 1148, 114C and 214A, 214B, 214C is visually distinguishable from at least the, or each, nearest, or neighbouring, other body segment 114A, 114B, 114C and 214A, 214B, 214C, and preferably from each other body segment 114A, 114B, 114C and 214A, 214B, 214C.

To this end, each body segment 114A, 114B, 114C and 214A, 214B, 214C has at least one visual indicator. The visual indicator may be provided by any one or more of the segment's colouring, patterning or texturing. In preferred embodiments, each body segment 114A, 114B, 114C and 214A, 214B, 214C is of a different colour. For example, the first segment 114A, 214A may be yellow, the second segment 114B, 214B may be red and the third segment 114C, 214C may be green. Alternatively, or in addition, each body segment 114A, 114B, 114C and 214A, 214B, 214C may be differently patterned, wherein different patterns may be formed by different shapes, colours and/or markings as is convenient. Alternatively or in addition, each body segment 114A, 114B, 114C and 214A, 214B, 214C may be differently textured, wherein different textures may comprise different formations in the body, e.g. differently shaped and/or arranged projections and/or recesses.

The visual indicator(s) may be provided in any convenient manner, during or after manufacture of the probe 12, 112, 212 and are provided on, or at least visible from, the external surface of the body 14, 114, 214. For example, the body 14, 114, 214 may be manufactured from materials, e.g. plastics, having the desired colours, or may have the desired visual indicators printed or painted thereon, or may have adhesive stickers applied thereto, or sleeves fitted thereon. In cases where the visual indicators are embedded in the body 14, 114, 214 are provided on an internal surface, the body should otherwise be transparent so that the visual indictors can be seen by the user. In any event, the visual indicators are discernible by the user looking at the body 14, 114, 214.

In preferred embodiments, the respective visual indicator(s) is provided along the entire respective body segment 114A, 1148, 114C and 214A, 214B, 214C. For example each body segment 114A, 1148, 114C and 214A, 214B, 214C may be appropriately coloured along its entire length. The colouring may be achieved using block colour or patterned colour, as desired.

The arrangement is such that, in use, a respective one of the body segments 114A, 114B, 114C and 214A, 214B, 214C is visible to the user at a designated location depending on the circumstances of the insertion. For example, in embodiments where the probe 12, 112, 212 is a nasogastric tube, the designated location is the ingress point 52 where the tube enters the patient's nostril, and different circumstances that may arise include (i) that the tube body 14, 114, 214 bends undesirably in the region of the patent's oral pharynx 54; (ii) that the tube body 14, 114, 214 enters the patient's trachea 56, in which case it will tend to bend in the region of the carina 58; or (iii) that the tube body 14, 114, 214 correctly reaches the patient's stomach 60.

In the embodiments of FIGS. 2 and 3, the tubes 112, 212 comprise a first body segment 114A, 214A, second body segment 1148, 214B and a third body segment 114C, 214C. The first body segment is closest to the leading end 120, 220 of the tube 112, 212, the third body segment is farthest from the leading end 120, 220, and the second body segment is located between the respective first and third body segments.

In these examples, two bend sensors 116A, 116B, 216A, 216B are shown. It is noted that, in alternative embodiments, only one bend sensor (or one set of bend sensors) is required, preferably located adjacent the leading end 120, 220 of the probe body 114, 214, for example at the location of the bend sensor 116A, 216A.

In the embodiment of FIG. 2, the first body segment 114A may for example be between 10 cm-20 cm in length, preferably approximately 15 cm in length. The second body segment 114B may for example be between 15 cm-25 cm in length, preferably approximately 20 cm in length. The third body segment 114C may for example be between 20 cm-30 cm in length, preferably approximately 25 cm in length. In this embodiment, the first, second and third body segments are 114A, 1148, 114C are contiguously arranged on the tube body 114, i.e. with no gaps between them.

In the embodiment of FIG. 3, the body segments 214A, 214B, 241C are longitudinally separated, i.e. there is a gap between each segment. In this example, the first body segment 214A may for example be located along the region between approximately 10 cm-20 cm from the leading end 220 of the tube body 214, the second body segment 214B may for example extend along the region between approximately 30 cm-40 cm from the leading end of the tube, and the third body segment 214C may extend along the region between approximately 55 cm-65 cm from the leading end of the tube.

In preferred embodiments, each of the first, second and third body segments 114A, 214A, 114B, 214B, 114C, 214C is provided with a different visual indictor, preferably a different colour.

It will be understood that the precise locations of the visually distinct body segments and/or their precise lengths may vary depending on the application and/or on the size of the patient (e.g. and adult or a child). Typically, however, each body segment is at least 2 cm long, preferably between 2 cm and 40 cm long, and most preferably between 5 cm and 30 cm long.

It will be understood that, in alternative embodiments, there may be only two visually distinct body segments, or there may be more than three visually distinct body segments. It is also noted that one or more body segment may be visually distinguishable from one or more other body segment without having to apply a visual indicator such as a colour or pattern; instead the natural characteristic(s) of the body may cause one or more segments of the body to be visually distinct over one or more other body segments that have been altered from the natural characteristic(s), e.g. a transparent segment of a tube is visually distinguishable from a segment that has been coloured.

For example, in alternative embodiments (not illustrated), the probe body may have only one body segment that is visually distinct from the rest of the body (thereby still creating at least two visually distinct body segments). This may be achieved by, for example, providing a colour or other visual indicator on said one body section and leaving the rest of the body in its natural state, or vice versa. In such an embodiment the (purposeful) visually distinct segment may correspond to the third segment 114C, 214C: if the user is informed of bending (or excessive bending (i.e. more than a threshold amount) or looping) at any time other than when this segment is visible at the nostril, then he may conclude that there has been an insertion failure.

It is noted that there may be one or more portion of the probe body that does not include a visual indicator specifically for the purpose of implementing the present invention but which is nonetheless visually distinguishable from one or more other portions or segments of the probe body. For example, in the embodiments of FIGS. 2 and 3, the tube portion between the end 119, 219 and the third body segment 114C, 214C, and/or the gaps between the body segments 214A, 214B, 214C may be visually distinguishable from the neighbouring body segments because, for example, they have the natural visual characteristics of whatever the tube is made from. This does not affect implementation of the invention since the arrangement of the length and/or longitudinal location of the purposefully visually distinct body segment(s) is such that (one of) the body segment(s) is visible to the user at a designated location (e.g. at the nostril or other ingress point) depending on a circumstance of the insertion. Hence, by correlating detected bends with whichever segment of the tube is protruding from the nostril (or other point of tube ingress), the user can determine if the tube has been correctly or incorrectly inserted.

Taking the embodiment of FIG. 2 by way of example, in use when the tip 122 of the tube 112 is 30 first inserted through a patient's nostril, a degree of bending is expected as it passes through the nose and into the oral pharynx. However, should the tip 122 become caught in the region of the oral pharynx 54, it may curl or loop in the patient's mouth. If so, the user will become aware of the undesired bending from the output device, e.g. the monitoring device 40, connected to the bend sensor(s) 116. For example, the monitoring device 40 may display a loop in this example. 35 Simultaneously, the user can see which segment of the tube 112 is exposed at the patient's nostril 52—in this case it will be the first segment 114A. These two pieces of information allow the user to determine that the insertion of the tube 112 has failed in the region of the oral pharynx and to act accordingly.

Should the tip 122 enter the trachea 56 it may curl or loop when it reaches the carina 58. If so, the user will become aware of the undesired bending from the output device, e.g. the monitoring device 40, connected to the bend sensor(s) 116. For example, the monitoring device 40 may display a loop or even just an unexpected bend in this example. Simultaneously, the user can see which segment of the tube 112 is exposed at the patient's nostril—in this case it will be the second segment 114B. These two pieces of information allow the user to determine that the insertion of the tube 112 has failed by entering the trachea to act accordingly.

When the tip 122 reaches the stomach 60 it will bend or curl. The user will become aware of this bending from the output device, e.g. the monitoring device 40, connected to the bend sensor(s) 16. Simultaneously, the user can see which segment of the tube 112 is exposed at the patient's nostril 52—in this case it will be the third segment 114C. These two pieces of information allow the user to determine that the insertion of the tube 112 is successful.

Extreme looping or curving would mean that tube could be curling in the oesophagus above the gastroesophageal junction and needs to be extracted partially until straight, and then reintroduced.

It will be seen that, in preferred embodiments, the system 10 is able to inform the user not only of the presence or otherwise of a bend, but also provide an indication of the severity of the bend and/or whether or not a loop is present. This bend information is advantageously provided in an image of the probe generated by monitoring device 40, but other types of output device may be configured to provide this information. In any event, the user is able to correlate the bend information with the visual information gained from observing which segment of the probe 12, 112, 212 is visible at the nostril, or other ingress point, in order to determine whether or not the insertion is being performed correctly.

The invention is not limited to the embodiment(s) described herein but can be amended or modified without departing from the scope of the present invention.

1. A probe insertion system comprising an elongate flexible probe having at least one bend sensor for detecting bends in at least one portion of the probe, the probe comprising a probe body having at least two longitudinally spaced, visually distinct body segments, the system further comprising at least one output device for rendering to a user an output indicative of at least one detected bend.

2. The probe insertion system of claim 1, wherein said at least one portion comprises a leading end portion.

3. The probe insertion system of claim 1, wherein at least two of said body segments, preferably all of said body segments, are arranged contiguously along the probe.

4. (canceled)

5. The probe insertion system of claim 1, wherein at least two of said body segments, preferably all of said body segments, are longitudinally separated.

6. (canceled)

7. The probe insertion system of claim 1, wherein each body segment is visually distinguishable from at least the, or each, nearest other body segment, each body segment preferably being visually distinguishable from each other segment.

8. (canceled)

9. The probe insertion system of claim 1, wherein at least one of said body segments is visually distinguishable by at least one visual indicator.

10. The probe insertion system of claim 1, wherein at least one of said body segments is visually distinguishable by its colouring.

11. The probe insertion system of claim 1, wherein at least one of said body segments is visually distinguishable by its patterning.

12. The probe insertion system of claim 1, wherein at least one of said body segments is visually distinguishable by its texturing.

13. The probe insertion system of claim 1, wherein at least one of said body segments is configured, by its position on the body and/or its length, for exposure at a patient ingress point in a designated insertion circumstance during use.

14. The probe insertion system of claim 1, wherein said at least one output device is configured to render to the user an output indicative of any one or more of: a detected presence of a bend; detection that a bend exceeds a threshold level; and/or detection of a loop in said body.

15. The probe insertion system of claim 1, wherein said at least one output device is configured to render to the user an output indicative of at least one bend detected in a leading end portion of the body.

16. The probe insertion system of claim 1, wherein said at least one output device is coupled to said at least one bend sensor, and wherein said at least one output device optionally comprises an audio, visual and/or haptic output device, and/or wherein said at least one output device optionally comprises a computing device, wherein said computing device is optionally configured to render an image of the probe including any detected bends.

17. (canceled)

18. (canceled)

19. (canceled)

20. The probe insertion system of claim 1, wherein each of said body segments is at least 2 cm long, preferably between 2 cm and 40 cm long, and most preferably between 5 cm and 30 cm long.

21. The probe insertion system of claim 1, wherein said at least two body segments comprise first, second and third body segments, said first segment being closest to a leading end of the probe, the third body segment being farthest from the leading end and the second body segment being between said first and third body segments, and wherein, preferably, said first body segment is between 10 cm-20 cm in length, preferably approximately 15 cm in length.

22. (canceled)

23. The probe insertion system of claim 21, wherein said second body segment is between 15 cm-25 cm in length, preferably approximately 20 cm in length.

24. The probe insertion system of claim 19, wherein said third body segment is between 20 cm-30 cm in length, preferably approximately 25 cm in length.

25. The probe insertion system of claim 21, wherein said first, second and third body segments are contiguously arranged on said probe.

26. The probe insertion system of claim 21, wherein at least part of said first body segment is located approximately 10 cm-20 cm from the leading end of the probe, and/or wherein at least part of said second body segment is located approximately 30 cm-40 cm from the leading end of the probe, and/or wherein at least part of said third body segment is located approximately 55 cm-65 cm from the leading end of the probe.

27. (canceled)

28. (canceled)

29. The probe insertion system of claim 1, wherein said probe is a nasogastric tube.

30. (canceled)
